(19) United States
(12) Patent Application Publication
(10) Pub. No.: US 2022/0334052 A1
(43) Pub. Date: Oct. 20, 2022

(54) ISOLATION AND CHARACTERIZATION OF PIGMENTS FROM CANNABIS

(71) Applicant: Alex Siegel, El Cerrito, CA (US)

(72) Inventor: Alex Siegel, El Cerrito, CA (US)

(21) Appl. No.: 17/720,385

(22) Filed: Apr. 14, 2022

Related U.S. Application Data
(60) Provisional application No. 63/174,976, filed on Apr. 14, 2021.

(51) Int. Cl. G01N 21/31; G01J 3/44; G01N 33/00

(52) U.S. Cl. CPC G01N 21/31; G01J 3/4406; G01N 33/0047

(57) ABSTRACT

A method of detecting cannabis pigments in a cannabis extract includes providing a sample of the cannabis extract, illuminating the sample with an excitation wavelength of ultraviolet light, detecting a fluorescence from the sample with a fluorescence detector, identifying carotenoid pigments by a fluorescence of 300 nm to 600 nm in the fluorescence spectrum, and identifying chlorophyll pigments by a fluorescence of 650 nm to 750 nm in the fluorescence spectrum.

### CROSS-REFERENCE TO RELATED APPLICATIONS

This application claims the benefit of priority to U.S. Provisional Patent Application No. 63/174,976, filed on Apr. 14, 2021, the contents of which are incorporated herein by reference in their entirety.

### FIELD

The present technology is generally related to detection and quantification of pigments in cannabis using spectroscopic techniques.

### SUMMARY

In one aspect, a method of detecting cannabis pigments in a cannabis extract is provided, the method including providing a sample of the cannabis extract, illuminating the sample with an excitation wavelength of ultraviolet light, detecting a fluorescence from the sample with a fluorescence detector, identifying carotenoid pigments by a fluorescence of 300 nm to 600 nm in the fluorescence spectrum, and identifying chlorophyll pigments by a fluorescence of 650 nm to 750 nm in the fluorescence spectrum. In some embodiments, the wavelength of ultraviolet light is about 280 to 550 nm. In some embodiments, the wavelength of ultraviolet light is about 300 to 510 nm. In some embodiments, the wavelength of ultraviolet light is about 320 nm to 400 nm. In some further embodiments, the wavelength of ultraviolet light is about 365 nm. In any of the above embodiments, the detecting a fluorescence may include monitoring fluorescence from the sample from a wavelength of about 300 nm to about 850 nm. In any of the above embodiments, the cannabinoids in the sample are not detected by the fluorescence detector. In any of the above embodiments, the method further includes identifying a fluorescence from 300 nm to 600 nm. In any of the above embodiments, the method may further include diluting the cannabis extract with an alkane. In such embodiments, the alkane may be hexane, heptane, octane, or nonane. The method may also include additional dilutions with additional alkane(s).

### BRIEF DESCRIPTION OF THE DRAWINGS

FIG. 1 is a fluorescence spectrum of a lutein pill (carotenoids/xanthophyll) with a cannabis oil sample, where the white is the cannabis oil and the black is the lutein, according to some embodiments.

FIG. 2 is a fluorescence spectrum of chlorophyll from a chlorella sample, according to various embodiments.

FIG. 3 is a fluorescence spectrum of highly concentrated lutein, which upon dilution would provide confirmation for the lutein in FIG. 1.

FIG. 4 is a fluorescence spectrum of a crude cannabis extract dissolved at about 0.1 g cannabis per 20 mL heptane, according to the examples.

FIG. 5 is a fluorescence spectrum, of a separated chlorophyll fraction from the cannabis dissolved at about 0.2 g per 20 mL heptane, according to the examples.

FIG. 6 is a fluorescence spectrum of a separated carotenoids fraction from the cannabis at about 0.03 g per 20 mL heptane, according to the examples.

FIG. 7 is an overlay of the carotenoid and chlorophyll fractions from FIGS. 5 and 6, for comparison to FIG. 4.

FIG. 8 is a fluorescence spectrum of a separated cannabinoids fraction from the cannabis extract at about 0.2 g per 20 mL heptane, according to the examples

FIG. 9 is a fluorescence spectrum of a separated cannabinoids fraction from the cannabis extract at about 2 g per 20 mL heptane, according to the examples

FIG. 10 is a fluorescence spectrum of a separated carotenoids fraction from the cannabis extract at a ratio of about 0.0015 g per 20 mL heptane, according to the examples.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Cannabis oil contains a diverse mixture of compounds, including a wide range of cannabinoid materials, and non-cannabinoid materials. As used here, the term “cannabinoid” is used to describe compounds that have a core structure based upon, and including that of THC (tetrahydrocannabinol). The family of cannabinoids includes, but is not limited to:

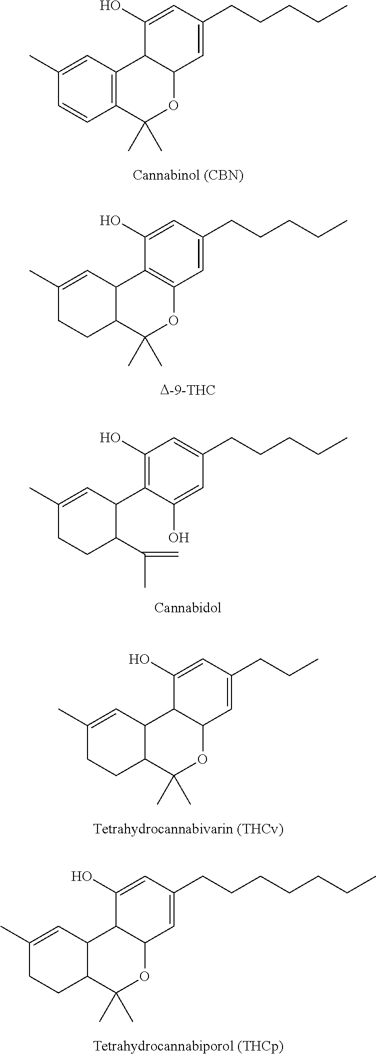

Other cannabinoids may include cannbigerol (CBG), cannabichromene (CBC), and cannbicyclol (CBL), as well as the acid counterparts to these such as canabidolic acid (CBDA), cannbinolic acid (CBNA), cannabigerolic acid (CBGA), cannabichromenic acid (CBCA), and tetrahydrocannabivarin acid (THCvA), and cannabicyclolic acid (CBLA).

Quantification of the cannabinoids present in cannabis oil is routinely accomplished in many cannabis-testing laboratories using high performance liquid chromatography (HPLC) paired with a photodiode (PD) array detector. And, quantification of non-cannabinoids in cannabis oil (i.e. terpenes, solvents, pesticides, heavy metals, and mycotoxins) is routinely done in many cannabis laboratories using mass spectrometry, inductively coupled plasma mass spectrometry, and with other similar tools to get a handle on the quality of the oil. Although solvents, pesticides, mycotoxins, and heavy metals, are important to quantify for consumer safety reasons, of the non-cannabinoids that are present in cannabis oil, the terpenes are typically the materials that are measured as an indicator of quality of the oil. The terpenes present in cannabis can account for 0.5 wt %, or more, of the finished product cannabis oil mass.

However, there is another class of non-cannabinoid components in cannabis oil, that are often as important as terpenes for determining quality, but which are not routinely determined. This other class of non-cannabinoids is known as the “pigments.” The pigments of cannabis oil are generally made up of carotenoids and chlorophylls, and some aged or oxidized cannabinoid samples may also contain significant amounts of oxidized carotenoids (xanthophylls). There may be significant chlorophyll breakdown products as well, such as chlorophyllide, pheophorbide, or any other porphyrin derivative relevant.

The ratio of cannabinoids and pigments in cannabis generally affects the ratio of cannabinoids and pigments in a resulting cannabis oil. The ratio of cannabinoids and pigments in crude cannabis oil is also affected by the crude oil extraction method, including differences in solvent polarity, temperature, duration, time, and pressure, and any processing done to the biomass prior to extraction of the oil.

Quick detection of pigments in cannabis oil, during or after processing, has traditionally relied on human sensory perception (e.g. color and taste). However, human sensory perception is, at best, inconsistent and incomplete because of contaminants, concentration differences, light conditions, crystallization, and more.

Use of an analytical instrument, rather than the human eye, for the detection of pigments during or after processing provides a more consistent and definite answer to the question of pigment content. However, there are no described, reliable methods of detecting these pigments in cannabis oil in a non-destructive manner and without the convolution by other cannabinoids and terpenes in the oil. Described herein is a novel method of detecting carotenoids and chlorophyll in cannabis oil in a non-destructive and near-instantaneous manner.

In terms of polarity, the pigments and cannabinoids present in cannabis oil are generally non-polar. However, in relative terms of polarity, based upon their water solubility, the following trend is observed.
Chlorophyll>cannabinoids>carotenoids

Accordingly, when a sample is tested and contains predominantly carotenoids, without (or with only trace amounts of) chlorophyll present, it may be assumed that the likely extraction method used in obtaining that sample of cannabis oil was with a non-polar extractant. Conversely, when a sample is found to contain higher levels of chlorophyll, with minimal or no carotenoids present, it may be assumed that the likely extraction method used in obtaining that sample of cannabis oil with a polar extractant or a warm and non-selective non-polar extractant. However, in almost all cases, some amount of both carotenoids and chlorophyll are detectable in cannabis oil samples due to the similar polarity of carotenoids, cannabinoids, and chlorophyll. The samples of cannabis oil may be diluted with an alkane (e.g. hexane, heptane, octane, etc) for the detection.

The present application provides methods for determining not only which pigments are present in a cannabis oil sample, but also their concentrations in the sample. The methods are applicable to any cannabis oil sample without regard to how it was obtained, whether through extraction, distillation, chromatography, membrane filtration, crystallization, color remediation powders, and the like.

The methods include using a fluorescence spectrophotometer having an excitation wavelength of 365 nm. The excitation light is then impinged on the sample, and the fluorescence spectrum is monitored at a wavelength of about 300 nm to about 800 nm. At an excitation wavelength of 365 nm, carotenoids are displayed to fluoresce between 300 nm and 600 nm. At an excitation wavelength of 365 nm, chlorophyll is displayed to fluoresce between 650 nm and 750 nm. Because of the different fluorescence behavior, the use of an excitation wavelength of 365 nm light allows for the simultaneous detection of carotenoids and chlorophyll in solution, even in the presence of cannabinoids. Other excitation wavelengths, 260 nm-500 nm for example, may exhibit a similar effect. Because the method of exciting with ultraviolet light and monitoring the fluorescence of the samples is a non-destructive method, it may be widely employed to cannabis oil or hemp oil without destroying the sample or needing to separate the molecules of interest from each other.

The concentrations at which the present disclosure is conducted attempt to mirror reasonable and standard manufacturing processes but may be adjusted by any, amount depending on pigment concentration of the extract sample. If the amount of pigmentation in an extract sample is acute, the amount of extract necessary in order to view the pigmentation using tools available increases. In general, extraction with a non-polar solvent is done with a volume of 1 liter of solvent per kilogram of cannabis. An industry standard expectation is that the extraction will yield 10% by mass from trim. This means that a concentration of 100 g cannabis extract per 1000 mL solvent is expected. At these concentrations, fluorescence interference peaks from the cannabinoids, when detecting and identifying the pigments are minimal to non-existent. When performing chromatography or other purification techniques, solvent volume may increase ten-fold one or more times, which may change the appearance of the fluorescent peaks. When non-processed crude extract samples are more concentrated than roughly 10% oil by mass, turbidity often prevents the accurate viewing of the pigments and results in a blank spectra reading with the tools available. Other tools may be able to handle such turbid solutions or filters may be able to remove certain particle contaminants to enhance viewing.

As used herein, “non-destructive” methods are those methods where at least 95 wt % of the pigmentation (or other desired component) in the tested sample is recoverable after the detection/determination is completed.

The samples may either be held in a stationary cuvette, one sample at a time, or the system may use a flow through cell in which the samples are subjected to excitation and fluorescence detection during the flow of the sample through the cell. This may allow for individual sampling of obtained oil products, or during production where the oils are tested before, during, and after isolation of the products from an extraction, distillation, or other process.

The present invention, thus generally described, will be understood more readily by reference to the following examples, which are provided by way of illustration and are not intended to be limiting of the present invention.

### Examples

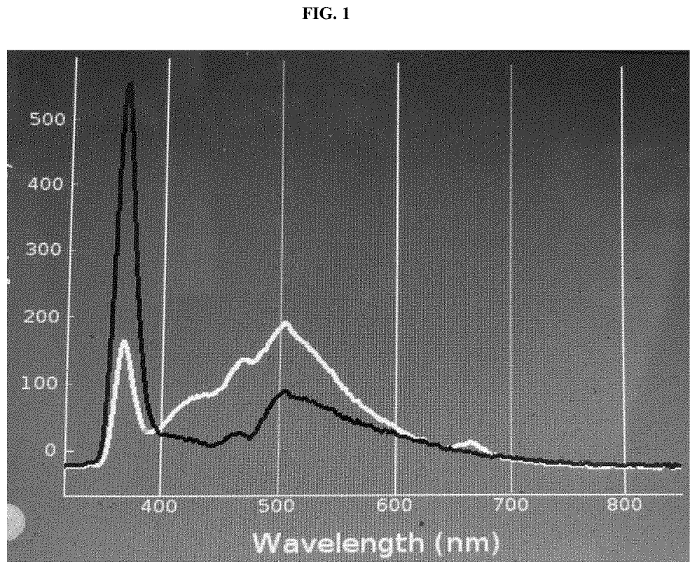

Example 1. Carotenoid determination. Concentrated carotenoid oil was purchased in the form of a synthetic supplement. The concentrated carotenoid oil was diluted at a ratio of one capsule (50 mg Lutein) to 20 mL heptane and sonicated at room temperature for 20 minutes to form Carot-D (the initial sample). The carotenoid sample was then further diluted with 1:1 heptane to form Carot-D2. Fluorescence spectra of Carot-D2 was then obtained from 300 nm to 800 nm using a 365 nm excitation wavelength. The spectrum is shown in FIG. 1 (black trace).

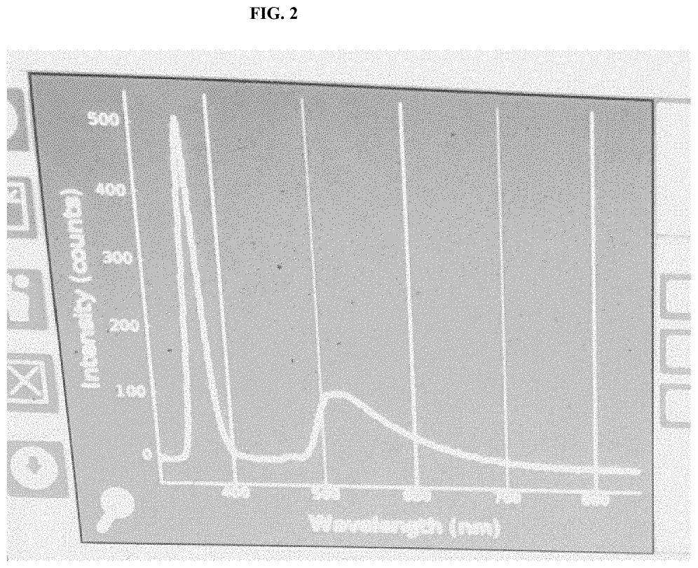

To display the similarity of the samples, also shown in FIG. 1, using an overlay feature, is the fluorescent peaks of a cannabis extract sample extracted with cold propane and diluted 0.2 g/20 mL. As can be seen by the trace, a chlorophyll peak is visible in the cannabis sample at around 600 nm. Concentration of carotenoids and chlorophyll greatly effects the exact peak shape and relative heights of the carotenoid fluorescence indicators, which in general contain three local maxima of slightly different height. However, as shown in FIG. 2, altering the concentration of the fluorescent species may completely change the shape of the fluorescent peaks, moving them slightly upstream or downstream. These changes in relative peak heights are indicators of changes in concentration of pigments. A reversal of the prominent peak in the chlorophyll region is also observed where highly concentrated samples exhibit a reversal in which of the two chlorophyll peak are dominant, although both peaks correlate with single species.

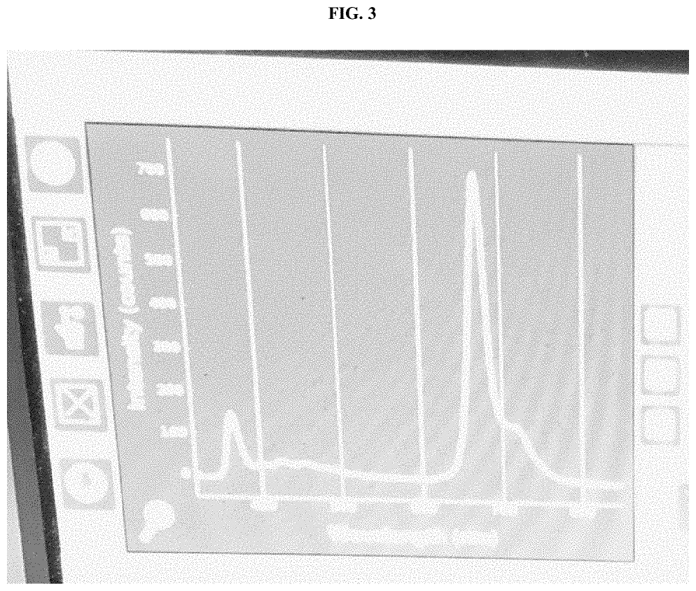

Example 2. Chlorophyll determination. Concentrated chlorophyll powder was purchased in the form of a “chlorella” powder supplement. To the chlorella powder (300 mg) was added heptane (20 mL), and the mixture sonicated at room temperature for 20 minutes to form Chloro-D1 (the initial sample). Chloro-D1 was then further diluted with 1:1 heptane to form Chloro-D2. Fluorescence spectra of Chloro-D2 was then obtained from 300 nm to 800 nm using a 365 nm excitation wavelength. The spectrum is shown in FIG. 3.

Example 3. A crude cannabis oil was obtained by extracting dried cannabis using heptane (a non-polar extraction solvent) at room temperature. The crude cannabis oil was concentrated in a rotary evaporator to remove the heptane, with a bath temperature on the rotary evaporator of about 40° C., to avoid, or at least minimize, the degradation of chlorophyll in the sample. The concentrated oil was then separated into three fractions using normal-phase chromatography. The stationary phase was silica 60 and the mobile phase was initially pure heptane during carotenoids collection but increased to up to 16% ethyl acetate and 84% heptane during cannabinoid and chlorophyll collection. Cannabis oil was loaded at a maximum of 25% by mass onto the silica. A pressure of 10 psi maximum was used. When higher pressure or higher loading is used, mixing of fractions becomes inevitable when using standard cannabis oil samples.

The first fraction to elute from the column contained carotenoids, the second fraction to elute contained cannabinoids, and the final fraction to elute contained chlorophyll.

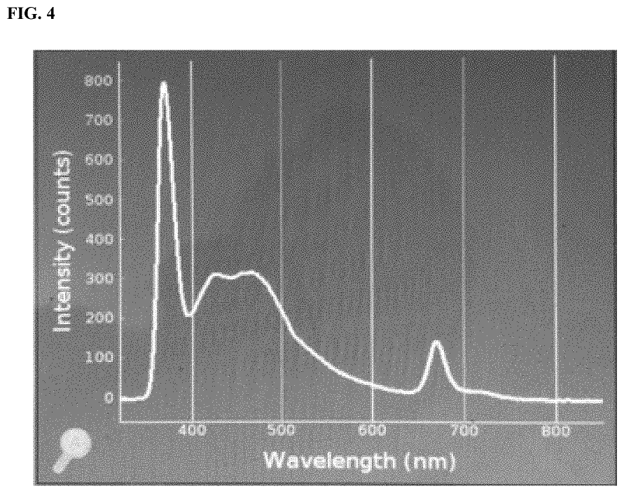

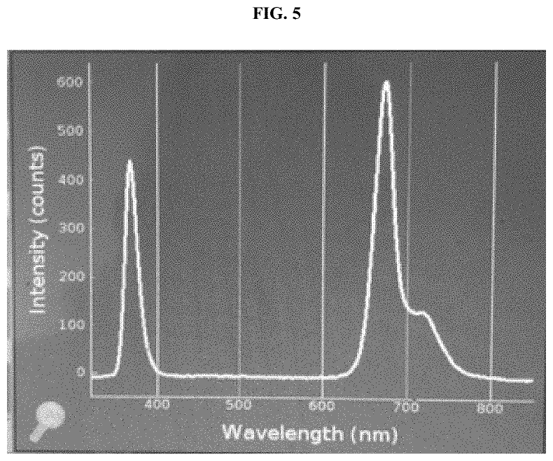

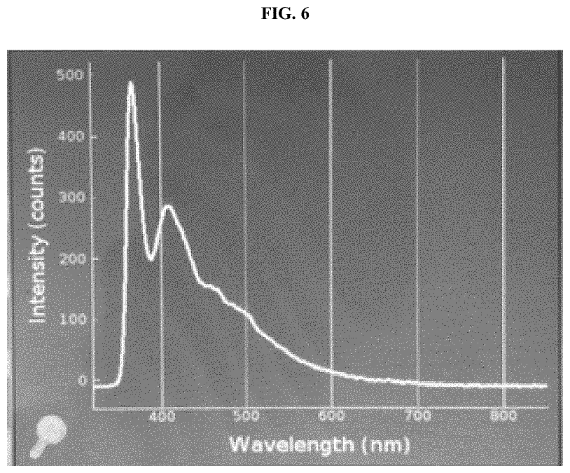

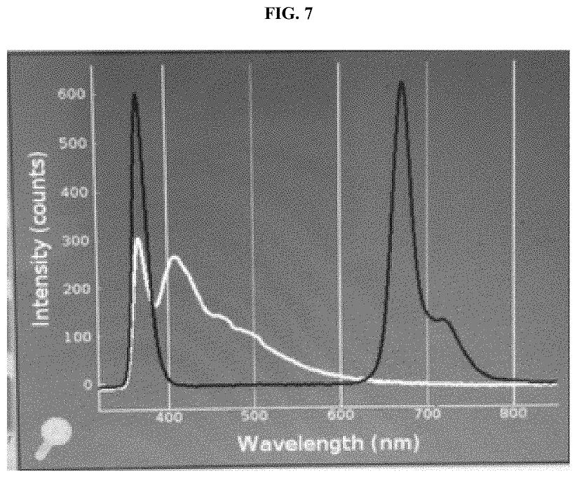

The three fractions were separately concentrated and then each was separately extracted into heptane at a maximum ratio of 0.2 grams concentrated pigment oil per 20 mL of heptane. Fluorescence spectra of each of the three fractions were obtained from 305 nm to 800 nm using a 365 nm excitation wavelength. The fluorescence spectrum of the crude cannabis extract is shown in FIG. 4, the fluorescence spectrum of the chlorophyll fraction is shown in FIG. 5, and the fluorescence spectrum of the carotenoid fraction is shown in FIG. 6. FIG. 7 is an overlay of FIGS. 5 and 6.

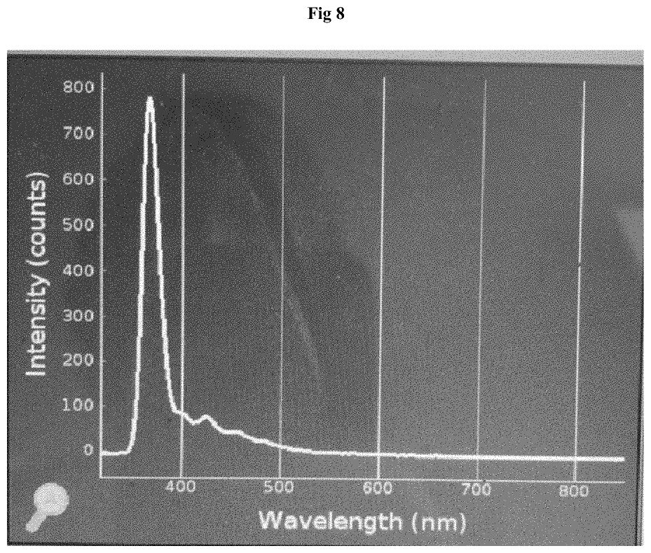

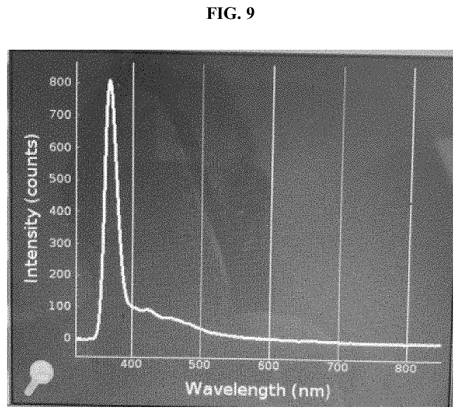

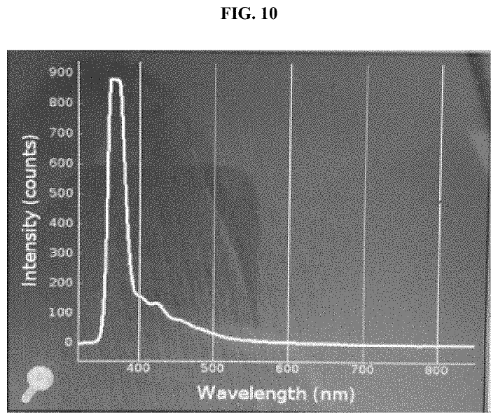

FIG. 8 is the fluorescence spectrum of the cannabinoid fraction at a concentration of 0.2 g concentrated cannabinoids per 20 mL heptane. FIG. 9 is the fluorescence spectrum of the cannabinoid fraction displayed in FIG. 8 at a concentration of 2 g concentrated cannabinoids per 20 mL heptane. As illustrated in the FIGS. 8 and 9 the presence of the cannabinoids did not significantly affect fluorescence readings at a wavelength of 365 nm, which seems to conflict with reported literature and industry practices. For example, in Bunn et al. (Cannabis Science and Technology 2(5):38-45 (2019)), multiple cannabis samples are analyzed and the results indicated that cannabinoids were generating significant fluorescent responses at these wavelengths. However, using our dilution protocol, though it is not necessary, generates a clear argument that the peaks seen are correlating to non-cannabinoid material. FIG. 10 represents a dilution of the carotenoids fraction an addition 1 mL Carot-D2 to 20 mL heptane. As a result, the spectrum obtained is nearly identical to both the dilute cannabinoid and concentrated cannabinoid spectra, indicating that the fluorescence in those fractions is a derivative of residual carotenoids, rather than the cannabinoids.

Example 4. Processing. After establishing the controls needed for the detection of carotenoids and chlorophylls in solution (see Examples 2 and 3), the techniques were applied to multiple manufacturing processes of cannabis oil. Trials included the use of powder filtration utilizing natural bentonites, silica, activated carbon, zeolite, alumina, and others. It was found that when using heptane as a filtration solvent, multiple powders including especially silica and activated carbon selectively removed most of the chlorophyll from the extract.

Trials also included the use of cannabinoid distillation, which demonstrated that green chlorophyll and red carotenoids can be separated from cannabinoids with distillation with some efficiency. AS-100 glassware available by request at Summit Research Inc and deep vacuum (30 micron at pump) were used to separate crude cannabis oil into three fractions. A volatiles fraction, which shows little fluorescence, a main body fraction, which contains some carotenoids and exhibits fluorescence as a result, and a tar fraction, which is nonvolatile and contains a large amount of chlorophyll and carotenoids.

A third pigment, which co-distills with THC, is visible in distilled cannabis and after removal of the two major pigments. This third pigment is likely to be composed of xanthophylls, which are carotenoid-like pigments, based upon their red color and increased polarity when compared to carotenoids.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of detecting cannabis pigments in a cannabis extract, the method comprising:
providing a sample of the cannabis extract;
illuminating the sample with an excitation wavelength of ultraviolet light;
detecting a fluorescence from the sample with a fluorescence detector;
identifying carotenoid pigments by a fluorescence of 300 nm to 600 nm in the fluorescence spectrum; and
identifying chlorophyll pigments by a fluorescence of 650 nm to 750 nm in the fluorescence spectrum.

2. The method of claim 1, wherein the wavelength of ultraviolet light is about 280 to 550 nm.

3. The method of claim 1, wherein the wavelength of ultraviolet light is about 320 nm to 400 nm.

4. The method of claim 2, wherein the wavelength of ultraviolet light is about 365 nm.

5. The method of claim 1, wherein the detecting a fluorescence comprises monitoring fluorescence from the sample from a wavelength of about 300 nm to about 850 nm.

6. The method of claim 1, wherein cannabinoids in the sample are not detected by the fluorescence detector.

7. The method of claim 1, further comprising diluting cannabis extract with an alkane.

8. The method of claim 7, wherein the alkane comprises hexane, heptane, octane, or nonane.
